(19) United States
(12) Patent Application Publication
(10) Pub. No.: US 2026/0235710 A1
(43) Pub. Date: Aug. 13, 2026

(54) SYSTEM AND METHOD FOR CORRECTIONS FOR SIMULTANEOUS COHERENT/INCOHERENT MOTION IMAGING

(71) Applicant: GE Precision Healthcare LLC, Waukesha, WI (US)

(72) Inventors: Isabelle Marie Heukensfeldt Jansen, Alplaus, NY (US); Nastaren Abad, Niskayuna, NY (US); Afis Ajala, Clifton Park, NY (US); Luca Marinelli, Schenectady, NY (US); Thomas Kwok-Fah Foo, Clifton Park, NY (US); Chitresh Bhushan, Schenectady, NY (US)

(21) Appl. No.: 19/052,541

(22) Filed: Feb. 13, 2025

(51) Int. Cl. G01R 33/56; A61B 5/00; A61B 5/055; G01R 33/563; G06T 5/73; G06T 7/38

(52) U.S. Cl. CPC G01R 33/5608; A61B 5/0042; A61B 5/055; G01R 33/56341; G06T 5/73; G06T 7/38; G06T 2207/10088; G06T 2207/30004

(57) ABSTRACT

A computer-implemented method for reconstructing slow-flow velocimetry data includes acquiring, via a processing system including one or more processors, diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having a fixed b-value and a fixed velocity encoding value configured to provide a velocity resolution of less than 1000 micrometers per second. The computer-implemented method also includes simultaneously determining, via the processing system, velocimetry metrics and diffusion tensor-derived metrics based on the diffusion-weighted MRI data.

### BACKGROUND

The subject matter disclosed herein relates to medical imaging and, more particularly, to a system and a method for simultaneous coherent/incoherent motion imaging (SCIMI).

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue partially align with this polarizing field, but precess about it at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

The study of cerebrospinal fluid motion in brain parenchyma, is of growing and significant interest to neuroscience as it is tied to clearance of metabolic waste products in the brain (glymphatic system). To date, the physiology and function of the glymphatic systems in humans remains elusive. Currently, non-contrast MRI is the most promising tool for noninvasive, qualitative imaging of slow glymphatic flow in vivo, in humans. While studies have been done in mice, there is currently no method that is sensitive enough to map physiologically-relevant fluid velocities in humans.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for reconstructing slow-flow velocimetry data is provided. The computer-implemented method includes acquiring, via a processing system including one or more processors, diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having both a fixed b-value and a fixed velocity encoding (VENC) configured to provide a velocity resolution of less than 1000 micrometers per second. The computer-implemented method also includes simultaneously determining, via the processing system, velocimetry metrics and diffusion tensor-derived metrics based on the diffusion-weighted MRI data.

In another embodiment, a system for reconstructing slow-flow velocimetry data is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. The actions include acquiring diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having both a fixed b-value and a fixed velocity encoding value configured to provide a velocity resolution of less than 1000 micrometers per second. The actions also include simultaneously determining velocimetry metrics and diffusion tensor-derived metrics based on the diffusion-weighted MRI data.

In a further embodiment, a non-transitory computer-readable medium, the computer-readable medium including processor-executable code that when executed by a processing system including one or more processors, causes the processing system to perform actions. The actions include acquiring diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having both a fixed b-value and a fixed velocity encoding value configured to provide a velocity resolution of less than 1000 micrometers per second. The actions also include simultaneously determining velocimetry metrics and diffusion tensor-derived metrics based on the diffusion-weighted MRI data.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique, in accordance with aspects of the present disclosure;

FIG. 2 illustrates a graph of a relative signal strength for the MAGNUS system in a b-VENC parameter space, in accordance with aspects of the present disclosure;

FIG. 3 illustrates a signal attenuation for the MAGNUS system for a constant VENC, in accordance with aspects of the present disclosure;

FIG. 4 illustrates gradient timing in the diffusion/velocity encoding portion of the pulse sequence for the MAGNUS system for a particular VENC, in accordance with aspects of the present disclosure;

FIG. 5 depicts MR images of a brain under different conditions utilized in determining the best resolution and operating point utilizing the disclosed sequence, in accordance with aspects of the present disclosure;

FIG. 6 depicts MR magnitude images obtained utilizing the disclosed sequence, in accordance with aspects of the present disclosure;

FIG. 7 illustrates a flow diagram of method for reconstructing slow-flow velocimetry data (SCIMI data pipeline), in accordance with aspects of the present disclosure;

FIG. 8 illustrates a schematic diagram of a SCIMI pipeline (e.g., process) for phase contrast reconstruction running in parallel to diffusion pipelines, in accordance with aspects of the present disclosure;

FIG. 9 depicts q-space spheres with corrections for gradient non-linearities of individual q-vectors, in accordance with aspects of the present disclosure;

FIG. 10 illustrates a schematic diagram of registration of complex images, in accordance with aspects of the present disclosure;

FIGS. 11 and 12 depict images of diffusion data and velocity data reconstructed from a single scan utilizing the disclosed sequence, in accordance with aspects of the present disclosure;

FIG. 13 depicts an example velocimetry output generated utilizing the disclosed technique, in accordance with aspects of the present disclosure; and

FIG. 14 depicts velocity streamlines over a cardiac cycle showing motion in parenchymal tissue generated utilizing the disclosed technique, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

The present disclosure provides systems and methods for reconstructing slow-flow velocimetry data. Slow velocimetry data has a velocity resolution of less than 1000 micrometers per second. For example, the velocity resolution may range from less than 1000 to greater than zero micrometers per second, between 900 to 100 micrometers per second, between 800 micrometers to 200 micrometers, between 700 micrometers to 300 micrometers, or between 600 micrometers and 400 micrometers. The velocity resolution may be any value between less than 1000 to greater than zero micrometers per second. In particular, a phase-sensitive diffusion MRI sequence is disclosed with a pulse timing sequence to achieve a velocity resolution of approximately 20 micrometers (μm)/second(s) and an integrated image reconstruction and velocity map generation pipeline. In particular, the disclosed techniques provide corrections for SCIMI. SCIMI uses a phase contrast imaging (PCI) sequence (or diffusion tensor imaging (DTI)) sequence to reconstruct complex images. The complex images are processed into two parallel streams: magnitude images for DTI processing, and phase images for a unique velocimetry. By simultaneously reconstructing magnitude and phase data, both metrics that characterize diffusive fluid (or tissue) motion and coherent velocity maps are calculated noninvasively in human subjects (e.g., time resolved over an entire cardiac cycle).

The use of a DTI pulse sequence (e.g., spin echo) creates velocity encoding (V_{ENC}) values on the order of approximately less than 1000 micrometers (μm)/second (s) (e.g., 300 μm/s) (in contrast to gradient echo-based velocimetry which is typically limited to 5 centimeters/s or greater). The pulse sequence is modified (e.g., increase distance between encoding phases) to maximize signal by using a “b-V_{ENC}” parameter space to create encoding values of b, V_{ENC}. Tissue specific constraints (e.g., of the brain's white matter and grey matter) to evaluate the signal strength's decay in this space and to create pulse sequences to maximize the signal. The disclosed pulse sequences break convention with regard to signal optimization via minimization of echo time (TE). Instead, the disclosed pulse sequences achieve better signals despite longer TE times. A disclosed reconstruction algorithm also accounts for gradient non-linearity, registration of complex images, background phase correction, and synchronization with periodic physiological signals (e.g., respiration or heartbeat) to map slow three-dimensional (3D) velocity vectors in tissue. The disclosed technique may be applied to different types of tissue. The disclosed technique facilitates research into assessment, diagnosis or intervention for traumatic brain injury, neurodegenerative disease, and brain health by providing in vivo imaging in humans of a biomarker previously only visible in animal studies. In particular, the disclosed technique enables measurement of in vivo velocities with a VENC below 1 mm/s without suffering from signal degradation.

The disclosed embodiments include providing a system and a method for reconstructing slow-flow velocimetry data (e.g., SCIMI data pipeline). The system and the method include acquiring, via a processing system including one or more processors, diffusion-weighted magnetic resonance imaging (MRI) data of a region of interest of a human subject with an MRI scanner utilizing a spin echo phase contrast imaging (PCI) sequence (e.g., spin echo diffusion tensor imaging echo planar imaging (DTI-EPI) sequence), wherein the spin echo PCI sequence has encoding pulses (e.g., sinusoidal encoding pulses, trapezoidal encoding pulses, etc.) having a fixed b-value and a fixed velocity encoding value (VENC) configured to provide a velocity resolution of less than 1000 micrometers per second. The system and the method also include simultaneously determining, via the processing system, velocimetry metrics and diffusion tensor-derived metrics based on the diffusion-weighted MRI data.

In certain embodiments, simultaneously determining the velocimetry metrics and the diffusion tensor-derived metrics includes generating, via the processing system, magnitude images and phase images from the diffusion-weighted MRI data. In certain embodiments, simultaneously determining the velocimetry metrics and the diffusion tensor-derived metrics further includes: performing, via the processing system, rigid body registration and eddy current correction on the magnitude images to create a forward map for each PCI volume, wherein the forward map is configured to translate a respective PCI volume to a common registered space and the forward map represents an original location of the respective PCI volume; and applying, via the processing system, respective forward maps to each respective PCI volume to generate registered magnitude volumes.

In certain embodiments, simultaneously determining the velocimetry metrics and the diffusion tensor-derived metrics further includes: creating, via the processing system, a tissue mask from the registered magnitude volumes; utilizing, via the processing system, the tissue mask to generate a reverse map for each PCI volume; applying, via the processing system, respective reverse maps to each PCI volume during phase unwrapping of complex diffusion-weighted images derived from the diffusion-weighted MRI data to generate the phase images; performing, via the processing system, background phase correction on the phase images to generate corrected phase images; and applying, via the processing system, respective forward maps to each respective PCI volume of each corrected phase image to generate registered phase volumes. In certain embodiments, simultaneously determining the velocimetry metrics and the diffusion tensor-derived metrics further includes: performing, via the processing system, gradient nonlinearity correction on the registered magnitude volumes to generate corrected q-space vectors for each PCI volume, wherein a magnitude and a direction are changed for corrected q-space vector for each PCI volume to account for the actual vs prescribed q-vector (accounting for both change in magnitude and direction of q-vectors) due to non-linearity off-isocenter of the MRI gradient coils; and performing, via the processing system, a linear fit utilizing the corrected q-space vectors in a respective q-space sphere for each PCI volume on the registered phase volumes to calculate velocity vectors for each PCI volume.

In certain embodiments, the system and the method include acquiring, via the processing system, periodic physiological signals from the human subject (e.g. electrocardiogram, respiratory waveform) simultaneously with acquisition of the diffusion-weighted MRI data, wherein the diffusion-weighted MRI data is acquired during an ungated, free-running scan. In certain embodiments, the system and the method include: utilizing, via the processing system, the periodic physiological signals to bin temporally each PCI volume into physiological bins, wherein each physiological bin includes an arbitrary grouping of multi-shelled q-space vectors and calculating, via the processing system, the velocity vectors for PCI volumes in each respective physiological bin of the physiological bins to generate to generate time-resolved velocity vectors over a physiological cycle (e.g. heart beat).

In the following disclosure, the disclosed techniques are described with regard to the brain. However, the disclosed techniques may be also utilized with other tissues where it is desired to analyze a fluid flow or tissue motion. Also, in the following disclosure, the disclosed techniques are described utilizing a spin echo DTI-EPI sequence. However, the disclosed techniques may be also utilized with other spin echo PCI sequences.

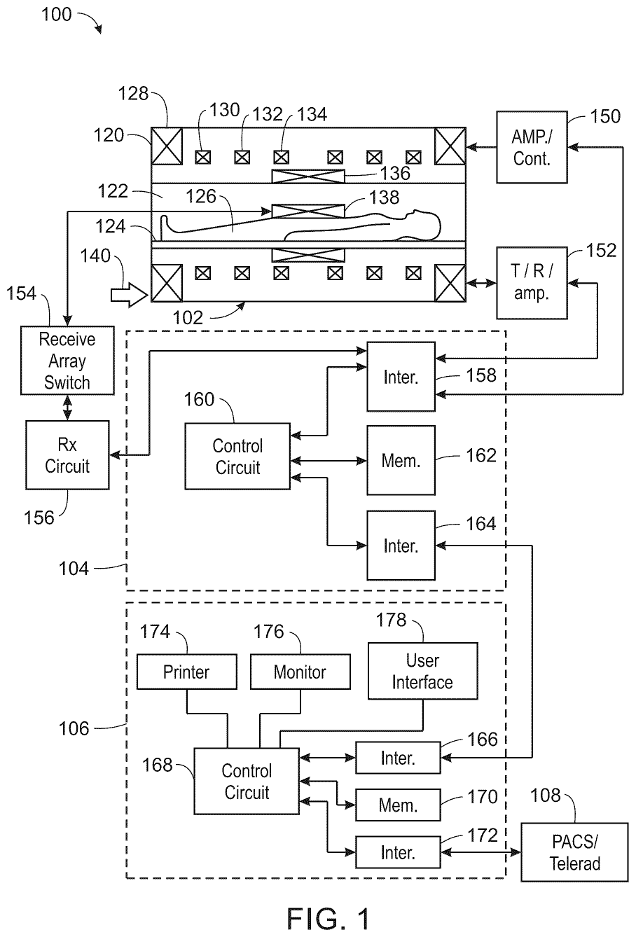

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more algorithms for reconstructing slow-flow velocimetry data (e.g., SCIMI data pipeline). In certain embodiments, the techniques disclosed herein may occur on a separate computing device having processing circuitry and memory circuitry.

A processing component (e.g., a microprocessor or processing circuitry) and a memory of the magnetic resonance imaging system 100, such as may be present in scanner control circuitry 104 and/or system control circuitry 106, may be used to execute stored software code, instructions, or routines for acquiring and processing the MR data. The term “code” or “software code” used herein refers to any instructions or set of instructions that control the magnetic resonance imaging system 100. The code or software code may exist in a computer-executable form, such as machine code, which is the set of instructions and data directly executed by the processing component of the scanner control circuitry 104 and/or system control circuitry 106, human-understandable form, such as source code, which may be compiled in order to be executed by the processing component of the scanner control circuitry 104 and/or system control circuitry 106, or an intermediate form, such as object code, which is produced by a compiler. In some embodiments, the magnetic resonance imaging system 100 may include a plurality of controllers.

As an example, the memory may store processor-executable software code or instructions (e.g., firmware or software), which are tangibly stored on a non-transitory computer readable medium. Additionally or alternatively, the memory may store data. As an example, the memory may include a volatile memory, such as random-access memory (RAM), and/or a nonvolatile memory, such as read-only memory (ROM), flash memory, a hard drive, or any other suitable optical, magnetic, or solid-state storage medium, or a combination thereof. Furthermore, processing component may include multiple microprocessors, one or more “general-purpose” microprocessors, one or more special-purpose microprocessors, and/or one or more application specific integrated circuits (ASICS), or some combination thereof. For example, the processing component may include one or more reduced instruction set (RISC) or complex instruction set (CISC) processors. The processing component may include multiple processors, and/or the memory may include multiple memory devices.

In certain embodiments (e.g., reconstructing slow-flow velocimetry data), the processing component (e.g., processing system including one or more processors) is configured to acquire diffusion-weighted magnetic resonance imaging (MRI) data of a region of interest of a human subject with an MRI scanner utilizing a spin echo phase contrast imaging (PCI) sequence (e.g., spin echo diffusion tensor imaging echo planar imaging (DTI-EPI) sequence), wherein the spin echo PCI sequence has encoding pulses having both a fixed b-value and a fixed velocity encoding value configured to provide a velocity resolution of less than 1000 micrometers per second. The processing component is also configured to simultaneously determine velocimetry metrics and diffusion tensor-derived metrics based on the diffusion-weighted MRI data.

In certain embodiments, simultaneously determining the velocimetry metrics and the diffusion tensor-derived metrics includes generating magnitude images and phase images from the diffusion-weighted MRI data. In certain embodiments, simultaneously determining the velocimetry metrics and the diffusion tensor-derived metrics further includes: performing rigid body registration and eddy current correction on the magnitude images to create a forward map for each PCI volume, wherein the forward map is configured to translate a respective PCI volume to a common registered space and the forward map represents an original location of the respective PCI volume; and applying respective forward maps to each respective PCI volume to generate registered magnitude volumes.

In certain embodiments, simultaneously determining the velocimetry metrics and the diffusion tensor-derived metrics further includes: creating a tissue mask from the registered magnitude volumes; utilizing, via the processing system, the tissue mask to generate a reverse map for each PCI volume; applying respective reverse maps to each PCI volume during phase unwrapping of complex diffusion-weighted images derived from the diffusion-weighted MRI data to generate the phase images; performing background phase correction on the phase images to generate corrected phase images; and applying respective forward maps to each respective PCI volume of each corrected phase image to generate registered phase volumes. In certain embodiments, simultaneously determining the velocimetry metrics and the diffusion tensor-derived metrics further includes: performing gradient nonlinearity correction on the registered magnitude volumes to generate corrected q-space vectors for each PCI volume, wherein a magnitude and a direction are changed for corrected q-space vector for each PCI volume to account for the actual vs prescribed q-vector due to non-linearity off isocenter of the MRI gradient coils; and performing a linear fit utilizing the corrected q-space vectors in a respective q-space sphere for each PCI volume on the registered phase volumes to calculate velocity vectors for each PCI volume.

In certain embodiments, the processing component is configured to acquire periodic physiological signals from the human subject simultaneously with acquisition of the diffusion-weighted MRI data, wherein the diffusion-weighted MRI data is acquired during an ungated, free-running scan. In certain embodiments, the processing system is configured to: utilize the periodic physiological signals to bin each PCI volume into temporal physiological bins, wherein each physiological bin includes an arbitrary grouping of multi-shelled q-space vectors and calculate the velocity vectors for PCI volumes in each respective physiological bin of the physiological bins to generate to generate time-resolved velocity vectors over a physiological cycle.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

DTI and gradient echo (GRE)-PC imaging defines characteristic values (b, VENC) for the measurement. Additionally, DTI sequences often acquire a large number of q-space directions distributed on a sphere to improve angular resolution, whereas PC imaging can be done with a minimum of 4 q-space directions to quantify 3-directional flow. The similarity in encoding schemes with additional redundant q-space information allows PC velocity images to be reconstructed from a DTI sequence. Where DTI reconstruction relies on the magnitude of the signal, PC reconstruction relies on the phase. Hence, if a DTI scan is reconstructed while preserving phase information, the resulting images can give information on the velocity. This allows both reconstruction pathways to be used with a single scan, simultaneously imaging coherent motion (velocity) and incoherent motion (diffusion).

With trapezoidal encoding pulses, the value of the encoding velocity, VENC, is determined by ν_{enc}=π/γGδΔ. Decreasing VENC requires increasing Δ or δ, which introduces practical restrictions when imaging in the brain. In phase contrast imaging based on gradient-echo, the VENC is limited by high-order concomitant field fields. Using a spin-echo DTI-EPI (echo planar imaging) sequence instead, it is not limited by such artifacts and the redundancy from measuring multiple extra q-space directions allows in-vivo measurement of velocities with VENC=300 μm/sec.

It is possible to design a sequence with both a fixed b-value and VENC in an appropriate range for motion in brain parenchymal tissue (<1000 μm/sec) by adjusting the timing parameters of the encoding gradients. For trapezoidal encoding gradients used in DTI-EPI, the shape of the encoding pulses is defined by the gradient strength G, the ramp time ζ, the width of a single trapezoid δ (area=Gδ), and time between the two lobes of the trapezoid Δ. In this configuration, the b-value and VENC are given by the following equations:

$$b = {γ}^{2} {G}^{2} [ {δ}^{2} ( Δ - \frac{\partial }{3} ) + {ζ}^{3} / 30 - \frac{{δξ}^{2}}{6} ] , \qquad ( 1 )$$

$${v}_{e n c} = \frac{π}{γ G δ Δ} . \qquad ( 2 )$$

Rearranging Equation 2 and substituting Δ=π/γGδν_{enc} into Equation 1 shows that values δ are found via roots of the equation:

$${δ}^{3} + ( \frac{{ξ}^{2}}{2} - \frac{3 π}{γ G {v}_{e n c}} ) δ + ( \frac{3 b}{{γ}^{2} {G}^{2}} - \frac{{ξ}^{3}}{10} ) = 0. \qquad ( 3 )$$

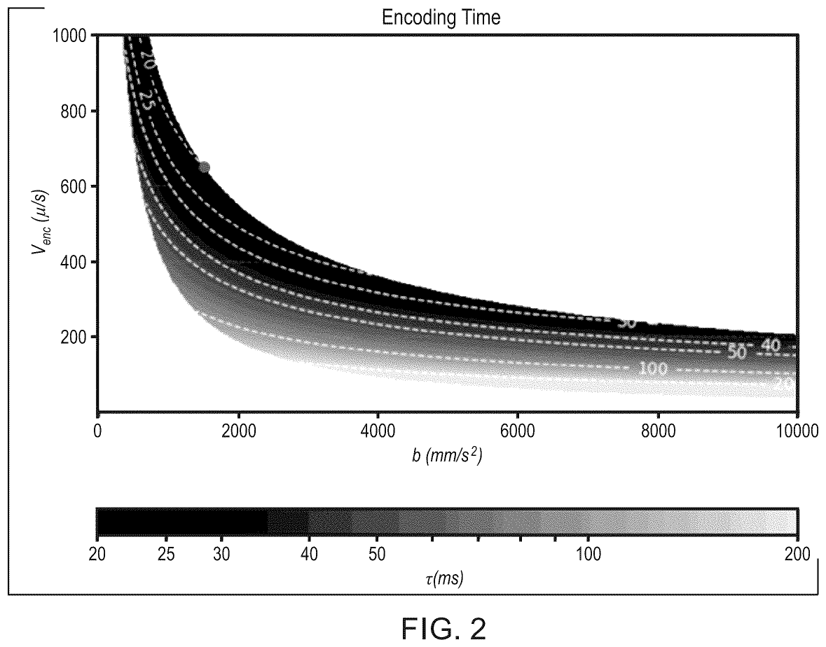

Once δ is determined, corresponding values of Δ can be calculated from Equation 2. This creates a parameter space for b-value and VENC based on system specifications such as gradient strength G, slew rate/ramp time ζ, and time needed for the 180° RF pulse t₁₈₀. Valid solutions require physical constraints to hold: the width of the encoding trapezoid must be longer than the ramp time (δ≥ζ) and Δ needs to be larger than the minimum time needed for one trapezoid and the RF 180 flip pulse (Δ≥δ+ζ+t₁₈₀). FIG. 2 shows the total encoding time τ=δ+Δ+ζ of the parameter space in a microstructure anatomy gradient for neuroimaging with ultrafast imaging (MAGNUS) system, a head-only gradient system for human scanning at 3T with a gradient strength of 300 mT/m and slew rate of 750 T/m/s. In particular, FIG. 2 provides the relative signal strength for the MAGNUS system in the b-VENC parameter space for a constant VENC. Due to the gradient strength and slew rate, the MAGNUS system provides access to a larger parameter space. It should be noted that parameter space is scanner-dependent.

Within this parameter space, we optimize the encoding pulses for a fixed VENC. When other acquisition parameters (e.g., field of view (FOV)) are held constant, the encoding time directly relates to TE, TE=τ(b, ν_{enc})+t(FOV). This allows signal attenuation in the image as a function of b to be determined from:

$$S = {S}_{0} {e}^{- D} {e}^{- r ( b , {v}_{enc} ) / {T}_{2}} . \qquad ( 4 )$$

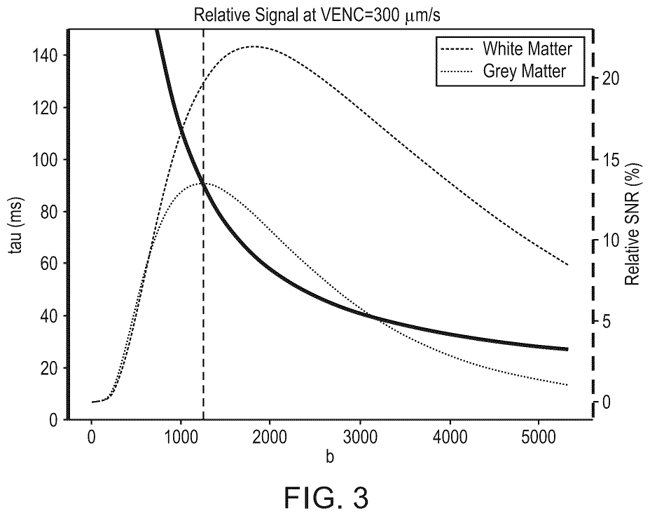

FIG. 3 shows the signal attenuation for the MAGNUS system for VENC=300 μm/s, using the reported apparent diffusion coefficients for the white matter and grey matter in the brain (e.g., D=4×10\hat{ }(−4) mm²/s and 8×10\hat{ }(−4) mm²/s). T2=80 ms and 90 ms for white matter and grey matter, respectively. In both white matter and grey matter, a lower b-value increases the contrast, even at the cost of a higher encoding time (indicated by the solid plot). As depicted in FIG. 3, the best combined contrast for both tissues occurs around b=1500 s/mm².

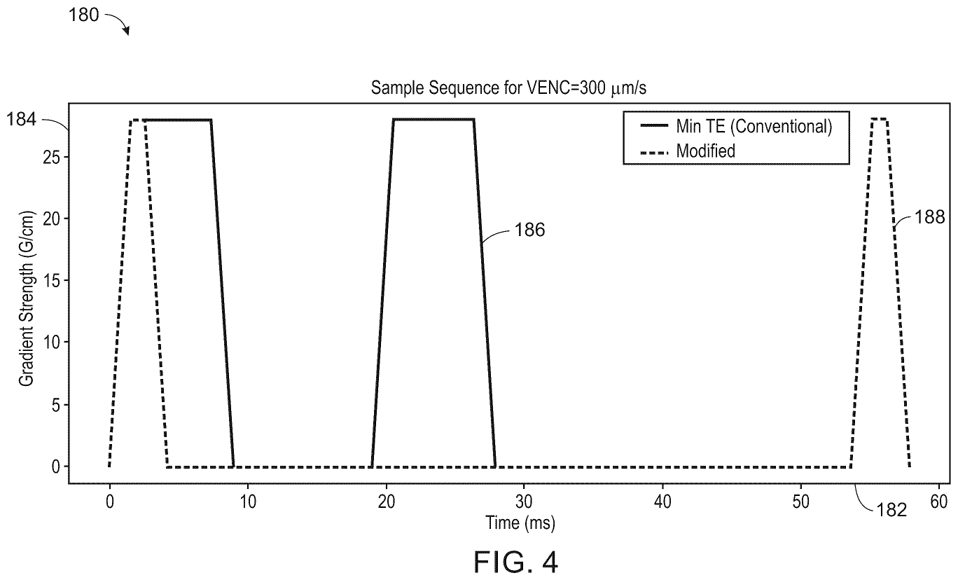

FIG. 4 depicts a graph 180 of a sample sequence for VENC=300 μm/s. Graph 180 includes an x-axis 182 representing gradient strength and a y-axis 184 representing time. Plot 186 represents a conventional TE and plot 188 represents a modified TE. With the modified TE (with an increased distance between encoding pulses), the pulses are further apart and skinnier. Increasing the distance between the encoding pulses for the purposes of recovering (maximizing) signal results in the lowering of b-value. As depicted, the encoding pulses have a trapezoidal shape. In other embodiments, the encoding pulses may have another shape (e.g., sinusoidal).

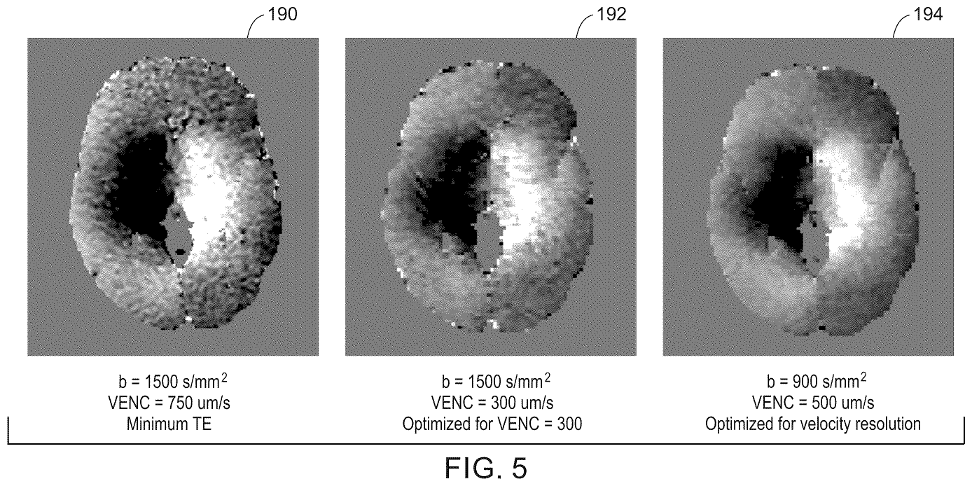

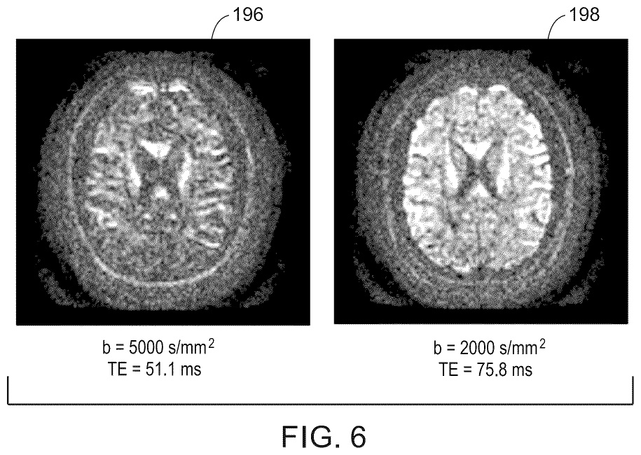

The VENC/Signal is utilized to determine the best resolution and operating point. FIG. 5 depicts MR images 190, 192, and 194 of a brain under different conditions are shown that were utilized in determining the best resolution and operating point utilizing the disclosed sequence. FIG. 6 depicts MR magnitude images 196 and 198 obtained utilizing the disclosed sequence. Image 198 despite having a longer TE has a better resolution.

To improve angular resolution, DTI sequences often use a larger number of q-space directions distributed in different arrangements, such as on a sphere or in multiple shells. Phase-contrast imaging typically uses conveniently chosen directions (e.g. +x and −x) such that phase images can be added or subtracted together to create velocity profiles. The velocity can still be solved in the over-determined system that using the DTI q-space sphere creates. This scheme therefore allows for simultaneous velocity and DTI metrics to be determined. The reconstruction of velocity vectors (νₓ, ν_{y}, ν_{z}) in this system expands the addition/subtraction of phase images into the form of a series of linear equations,

$$\hat{Q} \vec{v} = \frac{{v}_{e n c}}{π} Φ .$$

Expressed as matrices, the equation takes the form

$$[ \begin{matrix}\begin{matrix}{\hat{q}}_{1} & {\hat{q}}_{2}\end{matrix} & …\end{matrix} ] [ \begin{matrix}{v}_{x} \\ {v}_{y} \\ {v}_{z}\end{matrix} ] + {v}_{0} = \frac{{v}_{e n c}}{π} [ \begin{matrix}{ϕ}_{1} \\ {ϕ}_{2} \\ ⋮\end{matrix} ] , \qquad ( 5 )$$

where \hat{q}ₙ are the q-space unit vectors and v₀ represents the phase of a T2/b₀ image as background term. This form of the equation allows for the velocity profile to be calculated given the phases of an arbitrary set of q-space vectors, including multiple b₀ images or no b₀ image at all.

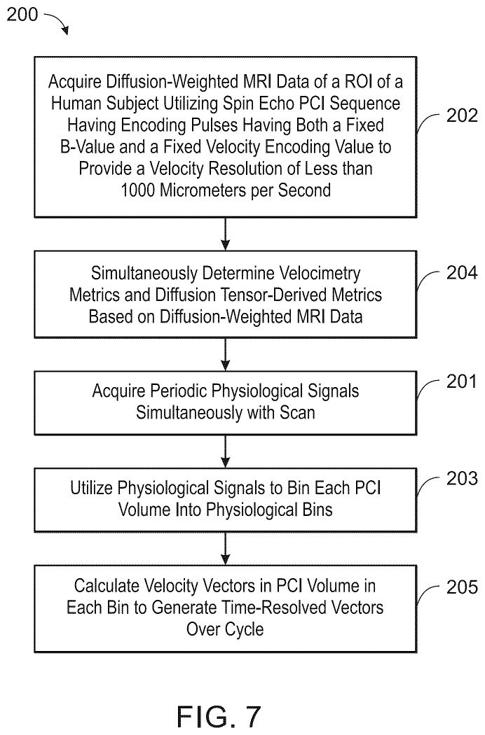

FIG. 7 illustrates a flow diagram of a method 200 for reconstructing slow-flow velocimetry (e.g., SCIMI data pipeline). One or more steps of the method 200 may be performed by processing circuitry of the magnetic resonance imaging system 100 in FIG. 1 or a remote computing device. One or more of the steps of the method 200 may be performed simultaneously and/or in a different order from that depicted in FIG. 7.

The method 200 includes acquiring diffusion-weighted magnetic resonance imaging (MRI) data of a region of interest (ROI) of a human subject with an MRI scanner utilizing a spin echo phase contrast imaging (PCI) sequence, wherein the spin echo PCI sequence has encoding pulses having both a fixed b-value and a fixed velocity encoding value configured to provide a velocity resolution of less than 1000 micrometers per second (block 202). Selection of a b-value and VENC determines the timing/spacing of the encoding pulses to achieve the desired velocity resolution. In certain embodiments, the spin echo phase PCI sequence is a spin echo DTI-EPI sequence. In certain embodiments, the diffusion-weighted data is acquired during a free-running (i.e., ungated, continuously running) scan over a dynamic, periodic physical process (e.g., heartbeat or respiration). Acquisitions are per slice (e.g., two-dimensional (2D) acquisition) and per volume (e.g., direction encoded: q-space). Alignment of acquisition over the physiological cycle (e.g., cardiac cycle) is pseudorandom due to natural variations (e.g., in each heartbeat). Acquisition time is slice-dependent. Each voxel (e.g., DTI or PCI volume) has a measurement from each volume and a time series for those measurements (e.g., based on normalized heartbeat). That time series may be distinct from neighboring voxels. As described in greater detail below, reconstruction may group several measurements together (e.g., via binning or sliding window) to find velocity. The method 200 also includes simultaneously determining velocimetry metrics and diffusion tensor-derived metrics based on the diffusion-weighted MRI data (block 204). In certain embodiments, the method 200 includes acquiring periodic physiological signals (e.g., heartbeat or respiration) from the human subject simultaneously with acquisition of the diffusion-weighted MRI data (block 201). As noted, in certain embodiments, the diffusion-weighted MRI data is acquired during a free-running scan. The method 200 also includes utilizing the periodic physiological signals to bin each PCI volume into physiological bins (block 203). Each physiological bin of the physiological bins comprises an arbitrary grouping of multi-shelled q-space vectors. The method 200 further includes calculating the velocity vectors for PCI volumes in each respective physiological bin of the physiological bins to generate to generate time-resolved vectors over a physiological cycle (block 205).

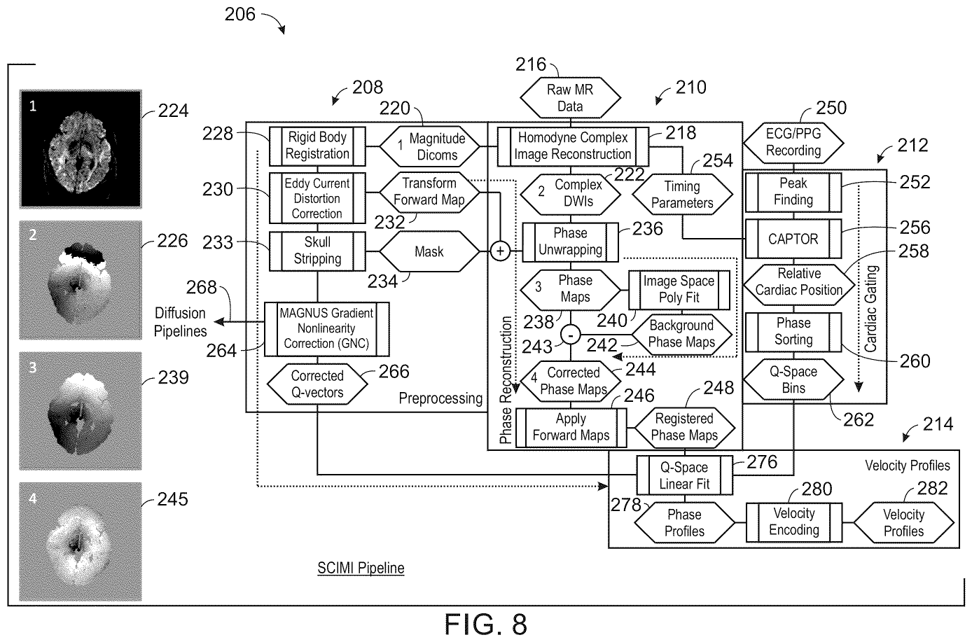

FIG. 8 illustrates a schematic diagram of a SCIMI pipeline (e.g., process 206) for phase contrast reconstruction running in parallel to diffusion pipelines. As depicted, retrospective cardiac gating is utilized in the process 206. The process 206 includes four stages: a magnitude-based preprocessing stage 208, a phase reconstruction 210 stage, a cardiac gating stage 212, and a velocity extraction stage 214. The process 206 includes reconstructing raw MRI data 216 (e.g., diffusion-weighted MRI data) (e.g., acquired utilizing the type of sequence (e.g., spin echo PCI sequence) disclosed herein) utilizing a homodyne reconstruction method (as indicated by reference numeral 218) to reconstruct a complete complex image preserving both amplitude and phase information. Magnitude images 220 (e.g., magnitude DICOMS) and complex diffusion-weighted images 222 (DWIs) are generated. Images 224, 226 are an example of a magnitude image and complex diffusion-weighted image, respectively.

As depicted in the magnitude-based processing stage 208, the magnitude images 220 are subjected to registration 228 (e.g., rigid body registration such as affine image registration) and linear eddy current distortion correction 230. For each volume (e.g., DTI volume) or voxel, the effects of registration and distortion correction are combined to create a forward map 232 (e.g., transform forward mask) indicating the original location of each voxel. When the tissue is the brain, the process 206 includes performing skull stripping 233 on the registered and distortion corrected magnitude images to generate a mask 234 (e.g., tissue mask).

In the phase reconstruction stage 210, the complex DWIs 222 are subjected to phase unwrapping 236 utilizing the tissue mask and forward map 232 to generate phase maps 238 (phase images). Image 239 is an example of a phase map. The phase reconstruction stage 210 includes performing image space polynomial fit 240 on the phase maps 238 to generate background phase maps 242. The background phase maps 242 are utilized in performing background phase correction 243 on the phase maps 238 to generate corrected phase maps 244. Image 245 is an example of a corrected phase map. The phase reconstruction stage 210 includes applying respective forward maps 232 (as indicated by reference numeral 246) to each respective PCI volume of each corrected phase image to generate registered phase maps 248 (and registered phase volumes).

As noted above, physiological signals from the human subject are acquired simultaneously with acquisition of the diffusion-weighted MRI data during a free-running scan. In certain embodiments, the heart rate is monitored to ensure the period does not fit evenly into the TR so that later retrospective gating would see q-space volumes distributed across the whole R-R interval instead of clustered. The cardiac gating stage 212 includes taking electrocardiogram (ECG) recordings/pulse plethysmograph (PPG) recording 250 and performing peak finding as indicated by reference numeral 252. During reconstruction, the acquisition time (timing parameters 254) for each slice is mapped onto the recorded ECG/PPG signal. The variability between the heart rate and the TR ensures that each slice has coverage over the entire cardiac cycle. In particular, cardiac phase to order reconstruction (CAPTOR) method is utilized (as indicated by reference numeral 256) to generate relative cardiac positions 258 (i.e., retrospective cardiac bins) each consisting of a subset of the total q-space acquired. This method determines systole and diastole separately and bins according to the relative percentage of each, allowing the bins to remain coherent through the end of diastole. Phase sorting 260 is performed to put the q-space volumes into corresponding bins to have different q-space bins 262. Each q-space bin 262 includes an arbitrary grouping of multi-shelled q-space vectors.

The magnitude-based processing stage 208 also includes performing gradient nonlinearity correction (GRC) (as indicated by reference numeral 264) on the distortion-corrected diffusion data (i.e., registered and distortion corrected magnitude images) to generate corrected q-space vectors 266 for each PCI volume. A magnitude and a direction are changed for corrected q-space vector for each PCI volume to account for a twist in direction. Distortion corrected and gradient non-linearity corrected diffusion data is utilized in conventional diffusion pipelines as indicated by arrow 268. In order to extract the simultaneous contrasts from these datasets, the magnitude component is further leveraged to generate simultaneous diffusion tensor-derived metrics. In addition to distortion corrects, the magnitude data is denoised with generalized spherical deconvolution (which models diffusion signal as a series of anisotropic and isotropic Gaussian compartments computed using the damped Richardson-Lucy algorithm. The coefficients are inserted into the forward model to derive a projection of the data for tensor fitting. Tuning the model parameters is important to the denoised approximation by generalized spherical deconvolution. For acquisitions on the MAGNUS platform, the tuned parameter space includes three anisotropic Gaussian compartments and a series of isotropic Gaussian compartments covering a broad range of diffusivities. Diffusion tensors were fit jointly by utilizing a non-negativity constrained linear least-squares approach.

GNC changes the magnitude and direction of q-space vectors on a voxel-wise basis, thus, accounting for errors introduced to twisting or rotation in direction of the q-space. As noted above, the scan may be free-running with physiological signals recorded for retrospective gating to sort the images into different bins according to a periodic signal (e.g., images might be sorted into 10 bins across the cardiac cycle). With natural variation in these signals, each bin may contain an arbitrary collection of images and their respective points in q-space.

The computation of the velocity vector has been expanded from the conventional form with fixed q-space points to allow for fits given an unknown assortment of q-space points using a least squares fits. These points may themselves be affected by the multi-shell acquisitions (for DTI) or gradient non-linearity, which is accounted for in the matrix design. A convex hull of all the points is created and the volume of the hull is compared to the volume of a tetrahedron created from a minimum number of required points to ensure a well-conditioned fit (avowing cluster of points). In certain embodiments, the surface area of the hull may be compared or a reference hull from a different set of points created.

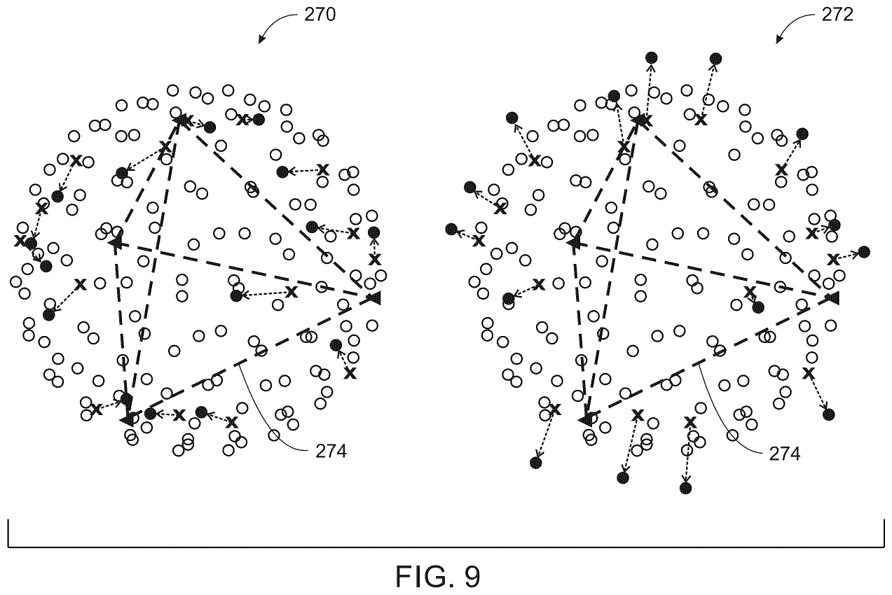

FIG. 9 depicts q-space spheres 270, 272 and the typical gradient non-linearities observed with MAGNUS gradients with imaging features furthest from isocenter experiencing significant decreases in gradient amplitude (greater than 15 percent). Gradient non-linearity effects on velocity encoding were corrected by first estimating the gradient field of each logical gradient axis using the tenth order spherical harmonic expansion. With the estimated field, the temporal evolution of voxel-wise VENC deviations are tracked (and adjusted) for parametric fitting. In the q-space speres 270, 272 is depicted the change in q-space in one cardiac bin due to gradient non-linearity from a gradient isocenter (x) to the edge of FOV along y (e.g., shown in q-space sphere 270) and z (e.g., shown in q-space sphere 272). Moving along y twists the vectors, while moving along z expands the vectors. Each vector may experience both a change and/or a twist around the origin by an angle θ. The specific points in the cardiac bin relate to the volume acquisition order, the TR, and the dynamic heart rate, creating selections with uneven distribution on the q-space sphere. For a well-conditioned fit, the vectors must cover as much q-space as a minimalist set of vectors, represented by the inner tetrahedron 274 in q-space spheres 270, 272 to create sufficient distribution in each direction. To evaluate the coverage, a convex hull was created from the unit vectors of each subset and the volume of the hull was compared to the volume of the tetrahedron 274 created from Hadamard encoding vectors representing minimum required coverage. A subset of vectors considered to span q-space sufficiently when the volume was greater than or equal to that of the representative hull.

Returning to FIG. 8, velocity extraction stage 214 includes performing a q-space linear fit (as indicated by reference numeral 276) utilizing the corrected q-vectors 266, the registered phase maps 248, and the q-space bins 262 to calculate 3D velocity vectors (phase profiles 278). In particular, the velocity extraction stage 217 includes performing a linear fit utilizing the corrected q-space vectors in a respective q-space sphere for each PCI volume on the registered phase volumes to calculate velocity vectors for each PCI volume. Velocity encoding 280 for each bin is performed to generate velocity profiles 282. The velocity profiles 282 (4D velocity profiles) provide time-resolved vectors over the cycle (velocities at different points of a cardiac cycle).

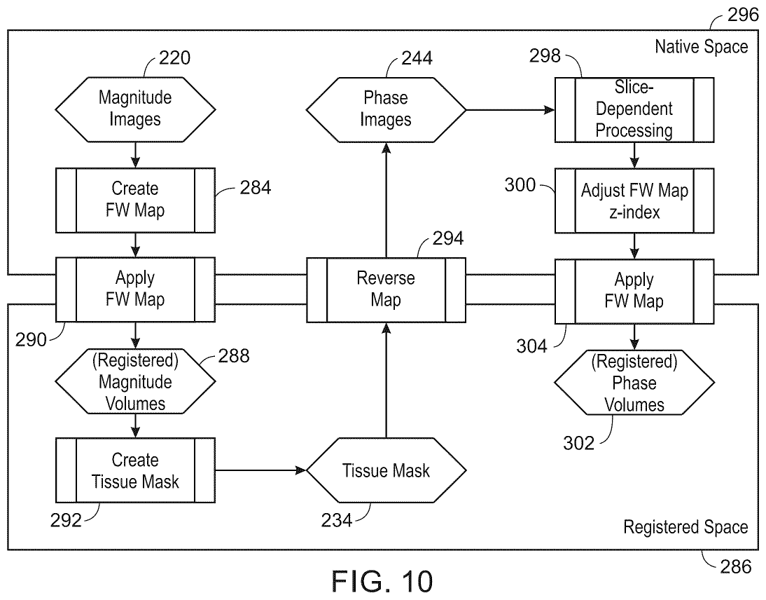

FIG. 10 illustrates a schematic diagram of registration of complex images. As depicted, magnitude images 220 are subjected to rigid body registration and eddy current correction on the magnitude images to create a forward map (after all corrections are determined) for each PCI volume as indicated by reference numeral 284. The forward map is created capturing all those corrections. The registration and distortion correction (and GNC calculation) are performed per volume. This disregards the discrete timepoints for each slice within the volume. The forward map 232 is configured to translate a respective PCI volume in and out of registered space 286 and the forward map represents an original location of the respective PCI volume. This forward map is modified to disallow inter-slice interpolations to preserve the time information. The forward map is applied to each respective PCI volume to generate registered magnitude volumes 288 as indicated by reference numeral 290. A tissue mask 234 is created from the registered magnitude volumes 286 as indicated by reference numeral 292. The tissue mask 234 is utilized to generate a reverse map for each PCI volume as indicated by reference numeral 294. Respective reverse maps are applied to each PCI volume (e.g., during phase unwrapping and background phase correction) to generate the phase images 244. The tissue mask is brought to each volumes native space 296 for slice-depending processing 298 that require a mask (e.g., during phase unwrapping and background phase correction). The z-index of the respective forward maps are adjusted (as indicated by reference numeral 300) to disallow interpolation between slices during registration to preserve the timing information distinct to each slice. Respective forward maps are applied to each respective PCI volume of each corrected phase image to generate registered phase volumes 302 and to complete the registration process as indicated by reference numeral 304.

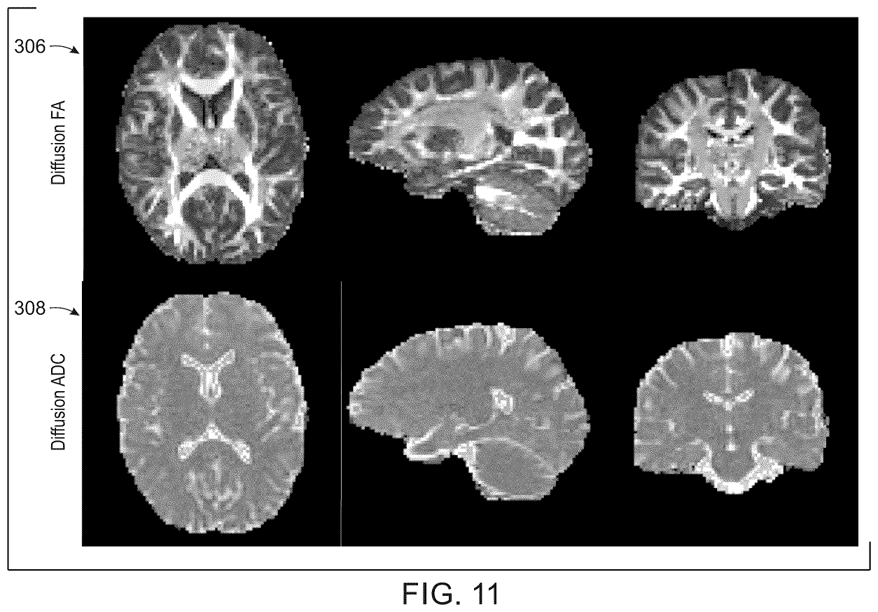

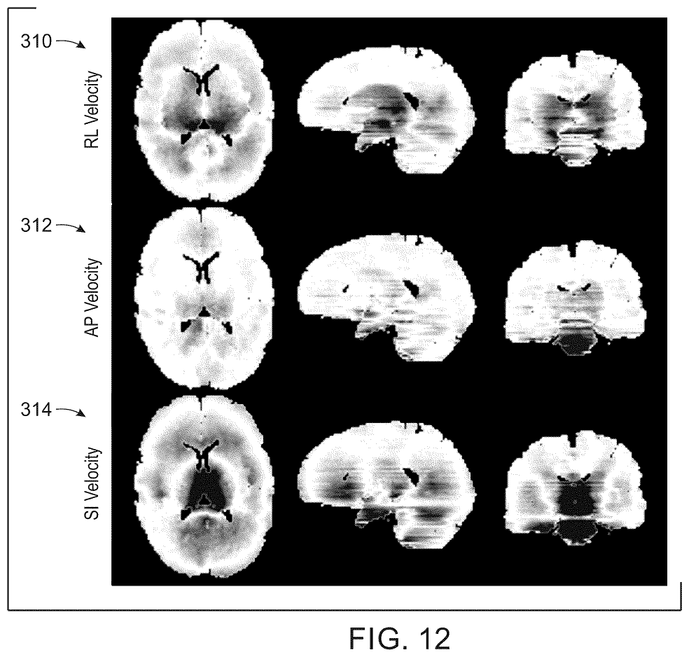

FIGS. 11 and 12 depict diffusion data and velocity data acquired simultaneously during a single scan. A top row 306 of images in FIG. 11 depicts axial, sagittal, and coronal views of a human subject's brain from a diffusion fractional anisotropy (FA) reconstruction. A bottom row 308 of images in FIG. 11 depicts axial, sagittal, and coronal views of the human subject's brain from a diffusion apparent diffusion coefficient (ADC) reconstruction. A top row 310 of images in FIG. 12 depicts axial, sagittal, and coronal views of the human subject's brain from a right-left (RL) velocity reconstruction. A middle row 312 of images in FIG. 12 depicts axial, sagittal, and coronal views of the human subject's brain from an anterior-posterior (AP) velocity reconstruction. A bottom row 314 of images in FIG. 12 depicts axial, sagittal, and coronal views of the human subject's brain from superior-inferior (SI) velocity reconstruction. Despite being processed only in a slice-wise fashion, you can see the axial, sagittal, and coronal views show coherent motion over the volume. FIGS. 11 and 12 demonstrate that a single scan can be utilized to image both diffusion through conventional processing pipelines and whole-brain, 4D vectors over the entire cardiac cycle.

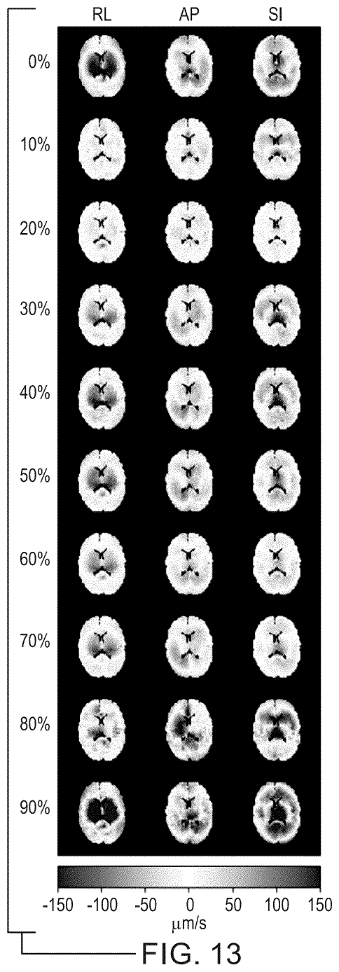

FIG. 13 depicts an example velocimetry output generated utilizing the disclosed technique. FIG. 13 depicts velocities in parenchymal tissue reconstructed in three dimensions shown as a percent of the cardiac cycle for a single human subject. FIG. 13 shows the three components of the velocity vector over the full cardiac cycle, shifting apparent peak systole to the end of the cardiac cycle (90%). During systole, motion is superior and laterally outwards from the ventricles. As the cardiac cycle moves into diastole, this motion is reversed, showing a pulsatile movement synchronized with the heart.

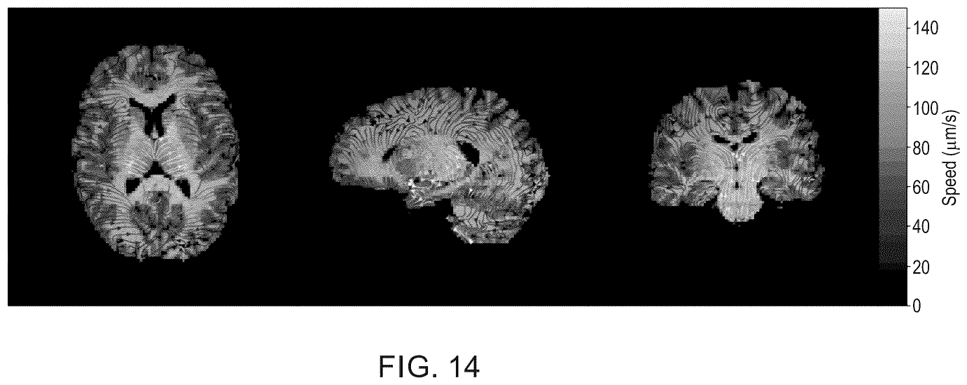

FIG. 14 depicts (e.g., a snapshot of) velocity streamlines over a cardiac cycle showing motion in parenchymal tissue generated utilizing the disclosed technique. Each timepoint is approximately 0.1 seconds (meaning movement down to five micrometers is captured (e.g., between 5 to 30 micrometers)). The velocity streamlines provide a view of the velocity maps and highlight the change in both direction and magnitude between stages of the cardiac cycle. Streamline colors are based on the total speed at each location, including out-of-plane speed.

Technical effects of the disclosed subject matter include facilitating research into assessment, diagnosis or intervention for traumatic brain injury, neurodegenerative disease, and brain health by providing in vivo imaging in humans of a biomarker previously only visible in animal studies. Technical effects of the disclosed subject matter include enabling measurement of in vivo velocities with a VENC below 1 mm/s without suffering from signal degradation.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for reconstructing slow-flow velocimetry data, comprising:
acquiring, via a processing system comprising one or more processors, diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having both a fixed b-value and a fixed velocity encoding value configured to provide a velocity resolution of less than 1000 micrometers per second; and
simultaneously determining, via the processing system, velocimetry metrics and diffusion tensor-derived metrics based on the diffusion-weighted MRI data.

2. The computer-implemented method of claim 1, wherein simultaneously determining the velocimetry metrics and the diffusion tensor-derived metrics comprises generating, via the processing system, magnitude images and phase images from the diffusion-weighted MRI data.

3. The computer-implemented method of claim 2, wherein simultaneously determining the velocimetry metrics and the diffusion tensor-derived metrics further comprises:
performing, via the processing system, rigid body registration and eddy current correction on the magnitude images to create a forward map for each PCI volume, wherein the forward map is configured to translate a respective PCI volume in and out of registered space and the forward map represents an original location of the respective PCI volume; and
applying, via the processing system, respective forward maps to each respective PCI volume to generate registered magnitude volumes.

4. The computer-implemented method of claim 3, wherein simultaneously determining the velocimetry metrics and the diffusion tensor-derived metrics further comprises:
creating, via the processing system, a tissue mask from the registered magnitude volumes;
utilizing, via the processing system, the tissue mask to generate a reverse map for each PCI volume;
applying, via the processing system, respective reverse maps to each PCI volume during phase unwrapping of complex diffusion-weighted images derived from the diffusion-weighted MRI data to generate the phase images;
performing, via the processing system, background phase correction on the phase images to generate corrected phase images; and
applying, via the processing system, respective forward maps to each respective PCI volume of each corrected phase image to generate registered phase volumes.

5. The computer-implemented method of claim 4, wherein simultaneously determining the velocimetry metrics and the diffusion tensor-derived metrics further comprises:
performing, via the processing system, gradient nonlinearity correction on the registered magnitude volumes to generate corrected q-space vectors for each PCI volume, wherein a magnitude and a direction are changed for corrected q-space vector for each PCI volume to account for a twist in direction; and
performing, via the processing system, a linear fit utilizing the corrected q-space vectors in a respective q-space sphere for each PCI volume on the registered phase volumes to calculate velocity vectors for each PCI volume.

6. The computer-implemented method of claim 5, further comprising acquiring, via the processing system, periodic physiological signals from the human subject simultaneously with acquisition of the diffusion-weighted MRI data, wherein the diffusion-weighted MRI data is acquired during a free-running ungated scan.

7. The computer-implemented method of claim 6, further comprising:
utilizing, via the processing system, the periodic physiological signals to bin each PCI volume into physiological bins, wherein each physiological bin comprises an arbitrary grouping of multi-shelled q-space vectors; and
calculating, via the processing system, the velocity vectors for PCI volumes in each respective physiological bin to generate time-resolved vectors over a physiological cycle.

8. A system for reconstructing slow-flow velocimetry data, comprising:
a memory encoding processor-executable routines; and
a processing system comprising one or more processors and configured to access the memory and to execute the processor-executable routines, wherein the processor-executable routines, when executed by the processing system, cause the processing system to:
acquire diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses both a fixed b-value and a fixed velocity encoding value configured to provide a velocity resolution of less than 1000 micrometers per second; and
simultaneously determine velocimetry metrics and diffusion tensor-derived metrics based on the diffusion-weighted MRI data.

9. The system of claim 8, wherein simultaneously determining the velocimetry metrics and the diffusion tensor-derived metrics comprises generating magnitude images and phase images from the diffusion-weighted MRI data.

10. The system of claim 9, wherein simultaneously determining the velocimetry metrics and the diffusion tensor-derived metrics further comprises:
performing rigid body registration and eddy current correction on the magnitude images to create a forward map for each PCI volume, wherein the forward map is configured to translate a respective PCI volume in and out of registered space and the forward map represents an original location of the respective PCI volume; and
applying respective forward maps to each respective PCI volume to generate registered magnitude volumes.

11. The system of claim 10, wherein simultaneously determining the velocimetry metrics and the tensor-derived metrics further comprises:
creating a tissue mask from the registered magnitude volumes;
utilizing the tissue mask to generate a reverse map for each PCI volume;
applying respective reverse maps to each PCI volume during phase unwrapping of complex diffusion-weighted images derived from the diffusion-weighted MRI data to generate the phase images;
performing background phase correction on the phase images to generate corrected phase images; and
applying respective forward maps to each respective PCI volume of each corrected phase image to generate registered phase volumes.

12. The system of claim 11, wherein simultaneously determining the velocimetry metrics and the diffusion tensor-derived metrics further comprises:
performing gradient nonlinearity correction on the registered magnitude volumes to generate corrected q-space vectors for each PCI volume, wherein a magnitude and a direction are changed for corrected q-space vector for each PCI volume to account for a twist in direction; and
performing a linear fit utilizing the corrected q-space vectors in a respective q-space sphere for each PCI volume on the registered phase volumes to calculate velocity vectors for each PCI volume.

13. The system of claim 12, wherein the processor-executable routines, when executed by the processing system, further cause the processing system to acquire periodic physiological signals from the human subject simultaneously with acquisition of the diffusion-weighted MRI data, wherein the diffusion-weighted MRI data is acquired during a free-running ungated scan.

14. The system of claim 13, wherein the processor-executable routines, when executed by the processing system, further cause the processing system to:
utilize the periodic physiological signals to bin each PCI volume into physiological bins, wherein each physiological bin comprises an arbitrary grouping of multi-shelled q-space vectors; and
calculate the velocity vectors for PCI volumes in each respective physiological bin to generate to generate time-resolved vectors over a physiological cycle.

15. A non-transitory computer-readable medium, the computer-readable medium comprising processor-executable code that when executed by a processing system comprising one or more processors, causes the processing system to:
acquire diffusion-weighted magnetic resonance imaging (MRI) data of a region of interest of a human subject with an MRI scanner utilizing a spin echo phase contrast imaging (PCI) sequence, wherein the spin echo PCI sequence has encoding pulses having a fixed b-value and a fixed velocity encoding value configured to provide a velocity resolution of less than 1000 micrometers per second; and
simultaneously determine velocimetry metrics and diffusion tensor-derived metrics based on the diffusion-weighted MRI data.

16. The non-transitory computer-readable medium of claim 15, wherein simultaneously determining the velocimetry metrics and the diffusion tensor-derived metrics comprises generating magnitude images and phase images from the diffusion-weighted MRI data.

17. The non-transitory computer-readable medium of claim 16, wherein simultaneously determining the velocimetry metrics and the diffusion tensor-derived metrics further comprises:
performing rigid body registration and eddy current correction on the magnitude images to create a forward map for each PCI volume, wherein the forward map is configured to translate a respective PCI volume in and out of registered space and the forward map represents an original location of the respective PCI volume; and
applying respective forward maps to each respective PCI volume to generate registered magnitude volumes.

18. The non-transitory computer-readable medium of claim 17, wherein simultaneously determining the velocimetry metrics and the diffusion tensor-derived metrics further comprises:
creating a tissue mask from the registered magnitude volumes;
utilizing the tissue mask to generate a reverse map for each PCI volume;
applying respective reverse maps to each PCI volume during phase unwrapping of complex diffusion-weighted images derived from the diffusion-weighted MRI data to generate the phase images;
performing background phase correction on the phase images to generate corrected phase images; and
applying respective forward maps to each respective PCI volume of each corrected phase image to generate registered phase volumes.

19. The non-transitory computer-readable medium of claim 18, wherein simultaneously determining the velocimetry metrics and the diffusion tensor-derived metrics further comprises:
performing gradient nonlinearity correction on the registered magnitude volumes to generate corrected q-space vectors for each PCI volume, wherein a magnitude and a direction are changed for corrected q-space vector for each PCI volume to account for a twist in direction; and
performing a linear fit utilizing the corrected q-space vectors in a respective q-space sphere for each PCI volume on the registered phase volumes to calculate velocity vectors for each PCI volume.

20. The non-transitory computer-readable medium of claim 19, wherein processor-executable code, when executed by a processing system, further causes the processing system to:
acquire periodic physiological signals from the human subject simultaneously with acquisition of the diffusion-weighted MRI data, wherein the diffusion-weighted MRI data is acquired during a free-running ungated scan;
utilize the periodic physiological signals to bin each PCI volume into physiological bins, wherein each physiological bin comprises an arbitrary grouping of multi-shelled q-space vectors; and
calculate the velocity vectors for PCI volumes in each respective physiological bin to generate to generate time-resolved vectors over a physiological cycle.
